(19) United States
(12) Patent Application Publication
(10) Pub. No.: US 2007/0014727 A1
(43) Pub. Date: Jan. 18, 2007

(54) Contrast media for nuclear spin tomography with use of the overhauser effect

(76) Inventors: Martin Rohrer, Berlin (DE); Alois Weidinger, Berlin (DE); Klaus-Peter Dinse, Heidelberg (DE); Andreas Hirsch, Rathsberg (DE); Wolfgang Harneit, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/353,228

(22) Filed: Feb. 14, 2006

Related U.S. Application Data
(60) Provisional application No. 60/653,488, filed on Feb. 17, 2005.

(30) Foreign Application Priority Data
Feb. 15, 2005 (DE) 102005007223.2

(51) Int. Cl. A61B 5/055

(52) U.S. Cl. 424/9.3

(57) ABSTRACT

The invention relates to contrast media for nuclear spin tomography (or else magnetic resonance tomography, MRT) with use of the Overhauser effect, which are suitable for transferring the magnetic orientation of electron spins to adjacent proton spins.

This application claims the benefit of the filing date of U.S. Provisional Application Ser. No. 60/653,488 filed Feb. 17, 2005 which is incorporated by reference herein.

The invention relates to contrast media for nuclear spin tomography (or else magnetic resonance tomography, MRT) with use of the Overhauser effect that are suitable for transferring the magnetic orientation of electron spins to adjacent proton spins.

### PRIOR ART

Nuclear spin tomography is an imaging process that uses the magnetic orientation of proton spins in a magnetic field that is applied from the outside. In the last two decades, MRT has developed into an established and widely used process in medical diagnosis. In contrast to other processes with comparable diagnostic importance—in particular computer tomography (CT)—no radiation exposure whatsoever or other physiological stress known to date for patients is produced with the MRT. With MRT, the proton spins of the hydrogen nuclei of tissue water (or of the blood, lymphatic system, etc.) are oriented by an externally strong magnetic field. Because of the low polarizability of the proton spins that depends on the strength of the applied static magnetic field, MRT devices with magnetic fields below 0.2 T have not been used in clinical routine. The most common field strength is now 1.5 T. By application of time-pulsed magnetic alternating fields whose carrier frequency corresponds to the resonance frequency of the proton spins in the respective field strength (for example 62 MHz at 1.5 T), a detection of proton spins is made possible, i.a., according to nuclear spin relaxation time, density and movement, from which conclusions on the type and condition of biological tissue are possible. By additional use of magnetic field gradients in various directions, a good spatial resolution in layered images or three-dimensional views can be produced.

The signal intensity and thus the image quality are determined in particular by the magnetic field strength, which is between 0.5 and 3 T in the case of a useful value for such devices.

The process for electron spins that is analogous to the magnetic resonance of proton spins is referred to as electron spin resonance (Electron Paramagnetic Resonance EPR). This effect had not been used previously for imaging in medical diagnosis. In the case of the Overhauser effect, a polarization transfer between various spin systems takes place. If this transition between electron spins and proton spins is carried out, the effect for the imaging is useful. This is described in, i.a., [ⁱ], [ⁱⁱ], [ⁱⁱⁱ] and [^{iv}]. In this connection, enhancement of the magnetic resonance by a polarization transfer of electron spins to the imaging nuclei surrounding them is carried out. This effect is based on the fact that electron spins can be more easily magnetically oriented than proton spins, and a transition of polarization to adjacent proton spins can be carried out. This is also referred to as dynamic nuclear spin polarization (Dynamic Nuclear Polarization—DNP) of the Overhauser type. The thus oriented proton spins can now be detected and evaluated with the common imaging processes, such as MRT. With the application in an imaging process, this results in an enhanced MRT signal. While in an ordinary nuclear spin tomography, magnetic fields of over 1 T are common, fields of only 10 mT are used in the case of a magnetic resonance tomography that is enhanced by means of the Overhauser effect, referred to in addition as OMRI (Overhauser-enhanced Magnetic Resonance Imaging). The magnetic field strength that is reduced by a factor of 100 compared to conventional MRT is necessary to produce excitation fields for electron spins, which are able to penetrate into deeper tissue layers despite the skin effect. With a magnetic field of 1 T, the electron spins have a resonance frequency of ˜10¹⁰ Hz. Such an excitation field, however, was already absorbed on the surface because of the skin effect and in this case would result in a hazardous heating of the tissue. The necessary reduction of the magnetic field randomly corresponds to the enhancement produced by the Overhauser effect, which is approximately at a factor of 100. Low-field systems are therefore obtained that have a good image resolution similar to that which is common in nuclear spin tomography but with considerable reduction of hardware, since superconducting magnets can be eliminated. This results in a more economical examination method. In clinical application, moreover, the tissue under study is exposed to significantly smaller magnetic fields, and the studies can be performed with devices that make possible free access to test subjects, i.e., patients with claustrophobia can also be treated easily. In addition, less discomfort from noise in comparison to conventional MRT is achieved.

In the case of nuclear spin tomography with use of the Overhauser effect (OMRI process), a (paramagnetic) substance whose electron spins are magnetically oriented by a magnetic field that is applied from the outside and that is suitable to transfer the magnetic orientation of the electron spins to adjacent proton spins must be supplied. The substances that were previously developed for OMRI are paramagnetic radicals. To date, however, a substance was neither subjected to a systematic, approval-relevant toxicological study nor brought into a first or later phase of a preclinical study or clinical development (there is no contrast medium approved for OMRI).

In EP 0 515 458, substances are described in which the free carbon radicals of triarylmethyl type (trityls) are used to produce a contrast medium for nuclear spin tomography with use of the Overhauser effect. EP 0 832 054 describes similar methyl radicals that are substituted by heterocyclic aromatic compounds for the same purpose.

Another substance class as a contrast medium for nuclear spin tomography with use of the Overhauser effect is described in EP 0 625 055 B1, according to which carbon allotropes and other analogous netlike molecular structures are used as basic structural components.

In WO 01/24696, a fullerol that has at least one free electron is indicated as a contrast medium for OMRI. This compound has a low stability, thus a transition of polarization can occur only during a relatively short period.

Relative to their suitability as OMRI contrast media, the above-mentioned triarylmethyl and triphenylmethyl radicals that are substituted in various ways have been described in the greatest detail in the recent literature (see, e.g., U.S. Pat. No. 5,289,125; U.S. Pat. No. 4,984,573; and EP 0 515 458).

The properties of these trityls, described in, for example, [i], [ii], [iii], [V] and [^{vi}] are therefore further described below as current “prior art.”

The above-mentioned substances make possible partially satisfactory in-vitro results [ii]; the previous in-vivo experiments on animal models show, however, an Overhauser enhancement that is too impractical to be suitable for general diagnostic imaging for clinical further development as an OMRI substance: in this respect, dosages on the order of between 1 mmol/kg of body weight and 5 mmol/kg of body weight are necessary [v], for which clinical development and approval is not promising from toxicological and commercial standpoints. Standard dosages of conventional contrast media for MRT are at most 0.1 mmol/kg of body weight, but they increase in the μmol/kg range. In addition, in the case of carbon radicals, a high degradation of the Overhauser enhancement is shown in plasma and in blood (compared to aqueous solutions), which can be attributed to reversible protein bonds of substances in blood and the effects of oxygen on the Overhauser effect [ii]. In deoxygenated water, the stability of different trityls with half-lives of between several hours and up to one year are indicated [ii], [iii]. In general, these radicals have a high chemical reactivity through the free electrons, which can result in an undesirable toxicity and in the loss of the effect.

The achievable Overhauser enhancement (at a given irradiated radiofrequency output for the electron spin transitions) depends on, i.a., the relaxation times of the electron spin.

In this case, long spin-lattice—(Tie) and spin-spin relaxation times (T₂ₑ) of the electron spin for OMRI contrast media are advantageous to achieve as high a polarization transition as possible during EPR excitation. The trityls are a relatively advantageous substance class in this respect, for which values on the order of T₁ₑ≈T₂ₑ≈8 μs (extrapolated on “infinite” dilution) were determined (from [ii] Table 3, p. 8, in isotonic NaCl solution at 37° C.: “predeuterated trityl”: T₁ₑ≅13 μs, T₂ₑ≅8 μs; “deuterated hydroxy trityl”: T₁ₑ≅11 μs, T₂ₑ≅8 μs, “symmetrical trityl”: T₁ₑ≅9 μs, T₁ₑ≅9 μs). Analogously to the comparatively long electronic relaxation times, narrow EPR resonance lines of this substance class are observed in liquid solutions. In [ii], EPR line widths of <1 μT in aqueous solution are indicated, in turn relative to infinite dilution, and in [i], a heterogeneously spread EPR line width of 6 μT is indicated. Both the EPR line widths and causally the electronic relaxation times greatly depend on the respective environmental conditions. These include in particular this concentration of contrast medium and oxygen dissolved in the environment. The relaxation times of the electron spin and the EPR line widths of the molecule are negatively influenced by intramolecular hyperfine structures and a large distribution of electronic spin density over the molecule. In this case, it is advantageous in the case of trityls that they have only one singular, dominant EPR line because of the deficient hyperfine structure of the unpaired electron with the central carbon atom (nuclear spin of ¹²C: I=O). In addition, with the proposed substances, a central localization of the spin density over a symmetrical molecular structure as well as a reduction of further intramolecular interactions by deuteration (substitution of hydrogen nuclei by the hydrogen isotope ²H) was desired. This was achieved to only a limited extent with the known approaches, however, and as the results suggest, in inadequate form.

Proton relaxivity, with which the shortening of the relaxation times of surrounding proton spins in the presence of contrast medium is described, represents another parameter that is of decisive importance for the suitability of the contrast medium both for OMRI and for conventional MRT. In this case, nuclear spin relaxation times of the protons that surround the OMRI contrast medium that are as short as possible are advantageous for a significant OMRI effect. This leads to the requirement for large values for proton relaxivity of the OMRI contrast medium. In the cited literature [i], [ii], and [iii], relaxivity values of between 0.14 mM⁻¹s⁻¹ and 0.44 mM⁻¹s⁻¹ are indicated for the trityl derivatives.

### DESCRIPTION OF THE INVENTION

The object of the invention consists in providing paramagnetic substances in which many electron spins are excited with the least possible energy and which are suitable for transferring this magnetic orientation by means of polarization transfer to as many nuclear spins as possible. They are to have long relaxation times of the electron spin and, following from this, very narrow and exactly limited EPR line widths.

In addition, for suitability as OMRI contrast media, the requirement of as efficient as possible a shortening of nuclear spin relaxation times of the surrounding protons exists. This is shown by as high a proton relaxivity as possible.

Moreover, they must be water-soluble, stable and toxically safe, so that they can be used as contrast media for OMRI in tissue studies on living organisms.

According to the invention, this object is achieved in that the paramagnetic substance consists of endohedral fullerenes that are filled with atomic nitrogen (N) or with atomic phosphorus (P).

The molecule cage for fullerenes consists of >60 carbon atoms. Thus, the endohedral fillerenes according to the invention can be used as contrast media for OMRI in tissue studies on living organisms, and the latter must be made water-soluble.

The solubilization of endohedral fullerenes is carried out in a preferred variant such that functional chemical groups that impart high water-solubility are covalently bonded to the fullerenes. The functionalization with the aid of malonic acid esters, which can be carried out analogously to the literature, is especially preferred [^{vii,viii,ix}]. These esters are then saponified [^{x}]. The carboxylic acid salts that are produced are very readily water-soluble.

The advantages of these compounds lie in their high stability and low toxicity.

This invention therefore relates to contrast media for nuclear spin tomography with use of the Overhauser effect (OMRI), characterized in that the contrast medium consists of endohedral fullerenes (Z@Cₓ)—Rₙ,

whereby Z means nitrogen or phosphorus,

R means a hydrophilic group,

n stands for a number between 1-10, and

X means a number between 60 and 82, as well as their physiologically compatible salts.

In a preferred embodiment, the endohedral fullerenes according to the invention are characterized in that
means a C(COY)₂ group, which is connected to the fullerene via two adjacent C atoms and thus forms a cyclopropane ring, and Y, independently of one another, means NR¹R² or OR¹, whereby R¹ and R², independently of one another, mean H, or C₁-C₁₀-alkyl, which is substituted with 1 to 6 hydroxyl groups, and n stands for a number 1-10,
   as well as their physiologically compatible salts.

In another preferred embodiment, the endohedral fullerenes according to the invention are characterized in that
R means a C(COY)₂ group, which is connected to the fullerene via two adjacent C atoms, and thus forms a cyclopropane ring,
Y means OR³, and
n is equal to 1,
whereby
   R³ is a dendrimeric branched alkyl radical that comprises up to 50 C atoms and that can be interrupted by up to 10 N or O atoms or —C(O)N(H) radicals and that can be substituted with up to 10 hydroxy, carboxylic acid or carboxylic acid amide groups, as well as their physiologically compatible salts.

In addition, this invention relates to a process for the production of water-soluble endohedral fullerenes according to the invention, characterized in that hydrophilic functional groups are coupled covalently to an endohedral fullerene (Z@Cₓ), whereby Z and x are defined as above.

Another variant for solubilization of the endohedral fullerenes consists in that the bare fullerenes are used as guests in a guest-host complex, whereby the host molecule is water-soluble to a high extent. For example, this complexing can be performed with cyclodextrin analogously to [^{xi}] In this case, in general no covalent bonds between guest and host molecules are produced. This variant has the advantage that the process that is used is simpler to perform. When using known host molecules, such as, for example, the cyclodextrins, the toxicological safety is already known.

The compounds according to the invention can easily be administered intravenously because of their water-solubility. There is also the possibility of depositing the contrast medium directly on defined sites to make possible a specific polarization exchange.

The advantages of the invention exist relative to the known contrast media for OMRI, i.a., in that these endohedral fullerenes are inert, i.e., chemically as well as electrically, their behavior is effectively neutral. The inclusion elements have stable shells such that no free electrons are located on the outside of the fullerene. It follows from this that these endohedral fullerenes affect their environment only via the magnetic dipolar action. In contrast to metal inclusions, which adhere to the inside of the cage, the inclusion elements are freely positioned in the middle of the cage of the fullerene, by which no interactions with the fullerene molecule take place. This symmetrical structure of the endohedral fullerene that is externally closed produces a high stability of this compound.

The substance N@C₆₃(COOH)₆ (III) according to the invention has long spin-relaxation times of the unpaired electron. It was possible to measure very long electronic relaxation times T₁ₑ≅150 μs and T₂ₑ≅22 μs in liquid solution, which are longer by at least one order of magnitude than that of the trityls, (see FIG. 4) with pulsed spin-echo EPR measurements in X-band (9.5 GHz EPR larmor frequency) and from CW EPR line widths in the L-band (1.1 GHz of EPR larmor frequency). From [ii] Table 3, page 8, in isotonic NaCl solution at 37° C.: “predeuterated trityl”: T₁ₑ≅13 μs, T₂ₑ≅8 μs; “deuterated hydroxy trityl”: T₁ₑ≅11 μs, T₂ₑ≅8 μs, “symmetrical trityl”: T₁ₑ≅9 μs, T₂ₑ≅9 μs.

The special suitability of the endohedral fullerenes according to the invention is shown in addition because of their surprisingly high relaxivity compared to the prior art that is determined in a standardized way by means of relaxometric measurements. Substance (III) has a relaxivity r₁≅47 mM⁻¹s⁻¹ (compared to the trityl derivatives with relaxivity values of between 0.14 mM⁻¹s⁻¹ and 0.44 mM⁻¹s⁻¹ [ii], and [iii]) in aqueous solution with a spin concentration of 6 μm at a solvent temperature of 37° C. This unexpectedly high value is present at a magnetic field strength of about 15 mT, which corresponds to a radiofrequency of 600 kHz and thus to the conditions of very low magnetic field strength that are necessary for OMRI.

Because of the spin relaxation times of the endohedral fullerenes according to the invention that are longer by at least a factor of 10 relative to the prior art, a significantly lower RF output for the excitation of EPR transitions is necessary with their use as OMRI contrast media. The desired enhancement of the MRT signals by the Overhauser effect with lower RF outputs for EPR excitation is therefore possible.

In addition, the proton relaxivity of the substances according to the invention, which, surprisingly enough, was noted to be significantly higher than that of the tritylene, leads to an additional, decisive increase of the OMRI effect: because of the shortening of the relaxation times of the surrounding proton spins, which is at least one order of magnitude greater compared to the tritylene, it is possible to perform OMRI studies with correspondingly fewer contrast medium dosages, in particular in the range starting from 0.05 mmol/kg of body weight up to 1 mmol/kg of body weight, in which no adequate OMRI effect can be achieved with the tritylene.

Since the oxygen-sensitivity of N@C₆₀ and derivatives (N@Cₓ)—Rₙ thereof is low and that of P@C₆₀ and derivatives (P@Cₓ)—Rₙ thereof is elevated, tomography images with and without oxygen detection (oximetry) for visualizing physiological or pathological conditions can be made by simultaneous measurements or measurements that are taken at different times with these two substances, similar to what was demonstrated [v], [vi] with tritylene by experiment with use of high contrast medium dosages (1 to 5 mmol/kg of body weight). With EPR measurements of P@C₆₀ (IX) in toluene, a reversible 20× widening of the EPR line after the solution is flushed with oxygen can be observed, which does not occur with N@C₆₀ and derivatives (see FIG. 4).

The invention therefore relates to the use of the water-soluble endohedral fullerene derivatives according to the invention, characterized in that first a contrast medium with atomic nitrogen as an inclusion element (N@Cₓ)—Rₙ is used, and a first nuclear spin tomography is performed, and then a contrast medium with atomic phosphorous as an inclusion element (P@Cₓ)—Rₙ is used at different times, and a second nuclear spin tomography is performed.

### BRIEF DESCRIPTION OF DRAWINGS

FIG. 1a: Diagrammatic visualization of an endohedral fullerene N@C₆₀ (I)

FIG. 1b: Diagrammatic visualization of an endohedral fullerene P@C₆₀ (IX)

FIG. 2: X-Band ESR spectrum of N@C₆₀ (I) in CH₂Cl₂ at 300 K

FIG. 3: L-Band ESR spectrum of N@C₆₃(COOH)₆ (III) in H₂O at 300 K

FIG. 4: FIG. 4 shows the reversible oxygen sensitivity of P@C₆₀ in toluene

FIG. 5: Diagrammatic drawing for synthesis of e,e,e-hexacarboxylic acid (IV) according to Example 1

FIG. 6: Diagrammatic drawing for synthesis of malonic acid precursors according to Example 2

FIG. 7: Synthesis of the dendrimer according to Example 2

FIG. 8: Diagrammatic drawing for synthesis of the dendrimer-fullerene according to Example 2

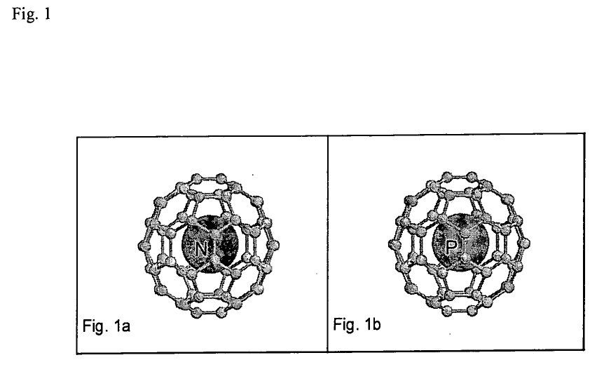

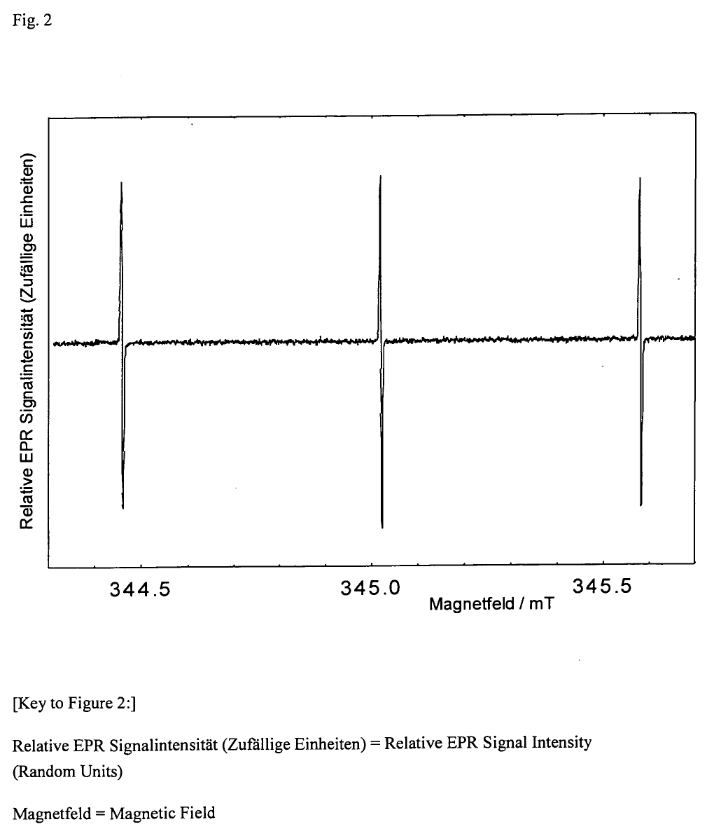

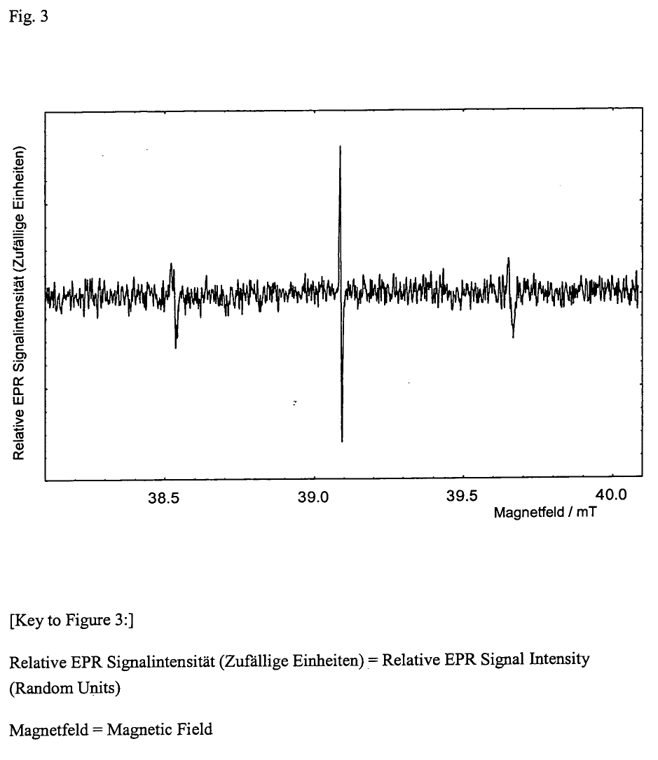

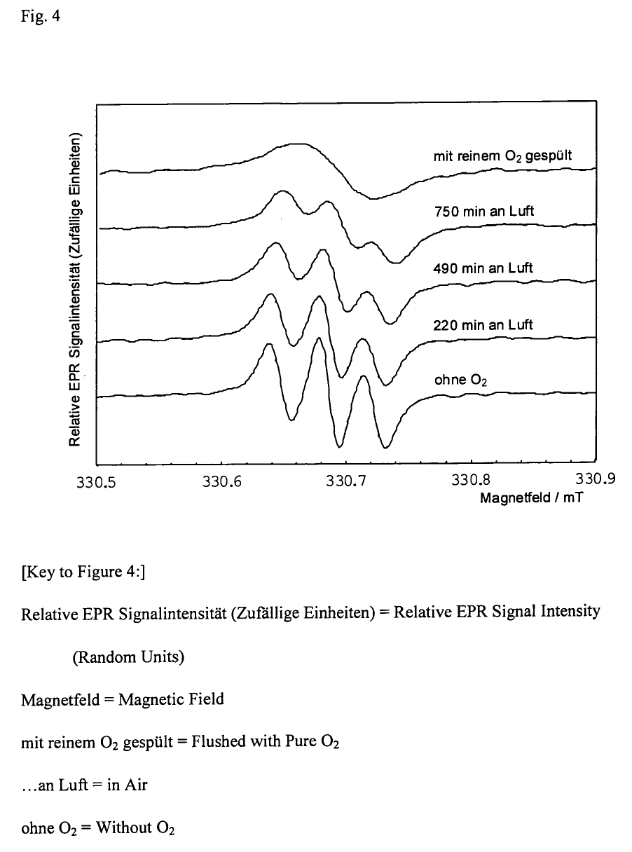

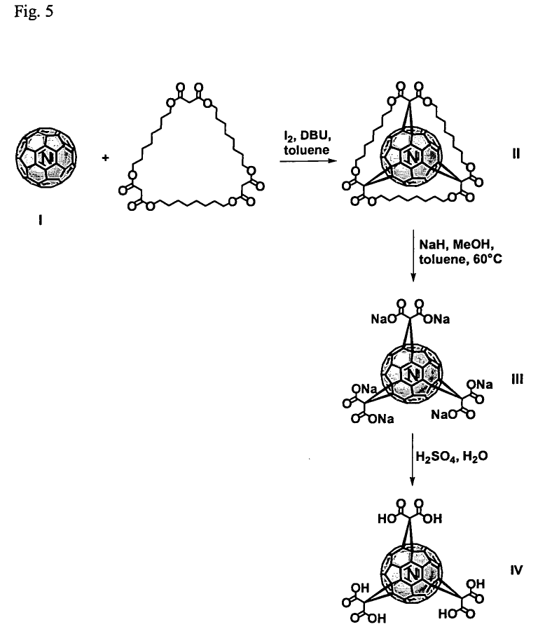

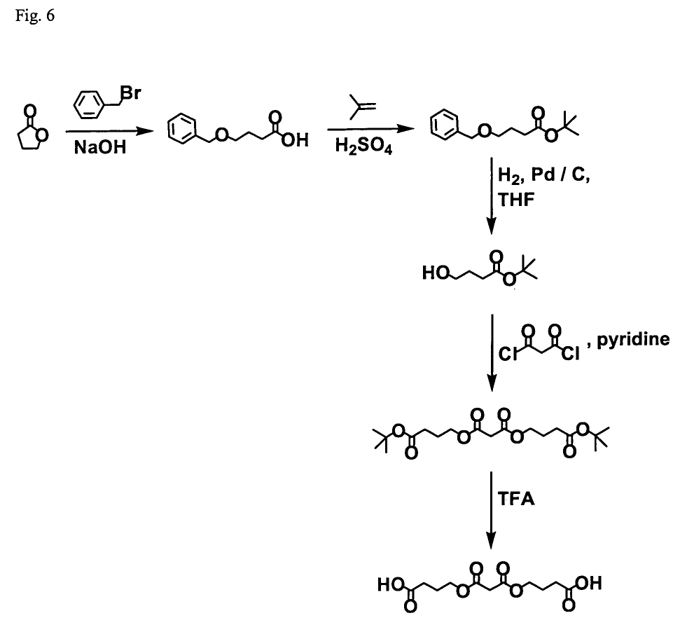

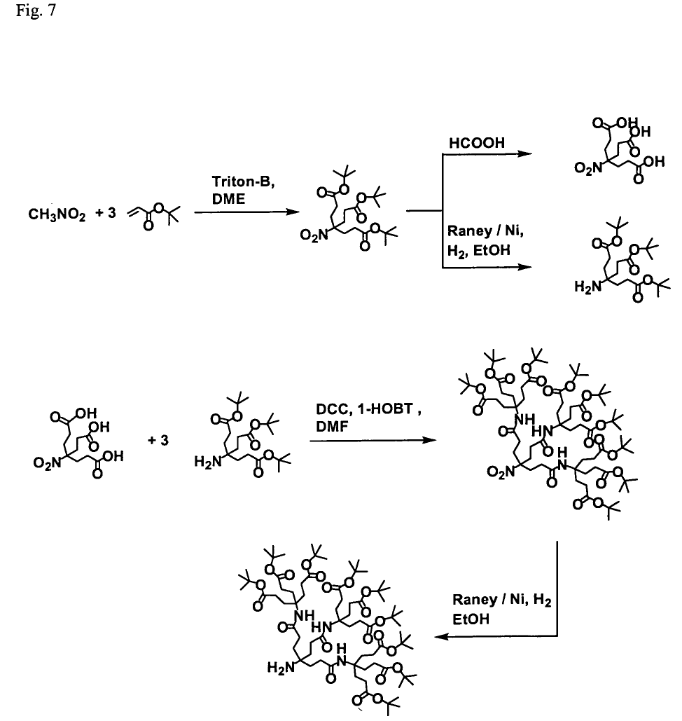

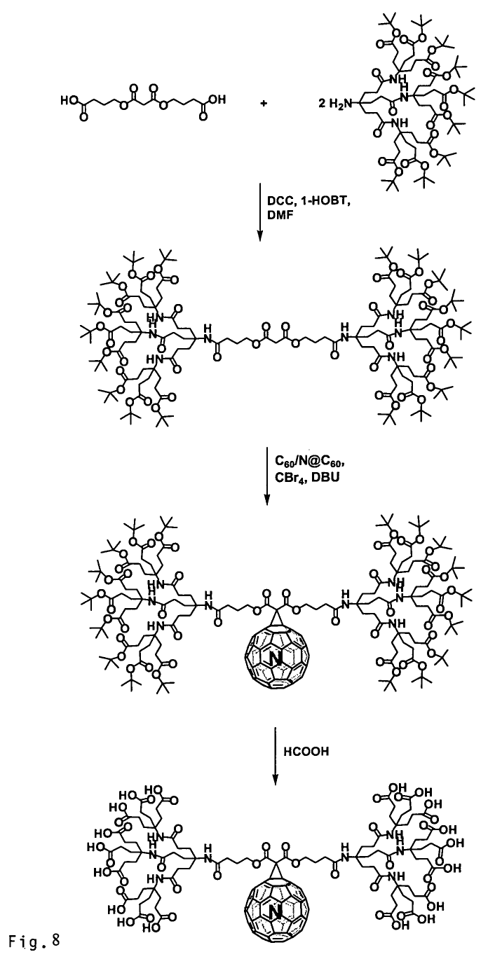

### EXAMPLES

The invention is to be explained in more detail below based on the embodiments.

The production of the endohedral nitrogen-fullerene N@C₆₀ (I) is carried out by, for example, ion implantation. [^{xii}]

The subsequent separation of the filled fullerenes from the empty fullerenes is carried out by High Performance Liquid Chromatography (HPLC) [^{xiii}].

All examples below are performed with a mixture that consists of N@C₆₀ and “empty” C₆₀. For the EPR and OMRI measurements, a spin content of between 10⁻² and 10⁻⁴ is sufficient. In the following, described examples, the spin content of the fullerene mixture is 10⁻⁴ to 10⁻².

### Example 1

### Synthesis of N@C₆₃ (COONa)₆ (III)

First, 255 mg (0.354 mmol/1.0 equivalent) of N@C₆₀ (I) is dissolved in 400 ml of dry toluene under argon cover gas and while being stirred. Then, 205 mg (0.319 mmol/0.9 equivalent) of cyclo-[3]-octylmalonate [^{xiv}] and 243 mg (0.956 mmol/2.7 equivalents) of iodine are added. A solution of 404 mg (397 μl/2.65 mmol/7.5 equivalents) of DBU in 160 ml of dry toluene can then be added in drops over 3 hours, whereby the color changes to deep orange. After another 10 minutes of stirring at room temperature, the crude mixture is subjected to flash chromatography on silica gel (6×25 cm). Traces of unreacted C₆₀ are eluted with other contaminants, first with toluene as a mobile solvent, and then the desired e,e,e-tris adduct (II) is eluted as an orange band with toluene/ethyl acetate (98:2). The e,e,e-(cyclo-[3]-octylmalonyl)-hexahydro-[60]-fullerene (II) is separated from the trans-4,trans-4,trans-4-isomer that is formed in traces by means of preparative HPLC on nucleosil (toluene/ethyl acetate 98:2), the fraction is concentrated by evaporation in a vacuum, and the product is precipitated from dichloromethane/pentane. After 3× washing with pentane and drying under high vacuum at 60° C., 174.0 mg (0.1282 mmol, 36.2%) of orange-red powder [e,e,e-(cyclo-[3]-octylmalonyl)-hexahydro-[60]fullerene (II) is obtained.

The e,e,e-tris-aduct-hexacarboxylic acid N@C63(COOH)₆ (IV) is obtained by a solution of 100 mg of the corresponding e,e,e-tris-adduct-malonate (II) in 50 ml of toluene being stirred for 3 hours under nitrogen as a cover gas in the presence of a 2033 molar excess of NaH at 60° C. Thus, the NaH is dispersed homogeneously into toluene. After 1 ml of methanol is added, the sodium salt of the e,e,e-tris-adduct-malonic acid (III) precipitates with vigorous gas generation quantitatively as a precipitate. The liquid phase is removed by centrifuging, and the precipitate is dried at 12 hours in a vacuum at 60° C.

The free acid (IV) can be obtained by washing the sodium salt of e,e,e-tris-adduct-malonic acid (III) with toluene, 2 M sulfuric acid and water and subsequent drying for 12 hours in a vacuum at 60° C.

### Example 2

### Synthesis of Dendrimer-Fullerene (VIII)

### 4-Benzyloxybutanoic Acid

129.9 g (2.32 mol) of finely ground 85% potassium hydroxide is added to a solution that consists of (44.3 ml, 50 g, 0.58 mol) of γ-butyrolactone and 276 ml (396.8 g, 2.32 mol) of benzyl bromide in 600 ml of toluene while being stirred, and it is refluxed with a water separator for 48 hours. The suspension that is obtained is mixed with 600 ml of water and 300 ml of diethyl ether. The aqueous phase is extracted three times with 300 ml each of diethyl ether. The collected organic phases are concentrated by evaporation and refluxed with 50 g of NaOH and 600 ml of water for another 20 hours. The aqueous phase is acidified with dilute sulfuric acid and then extracted three times with 300 ml each of CH₂Cl₂. After the organic phase is dried on MgSO₄ and after the solvent is distilled off, 24 g of a light yellow oil is obtained. With the solution that is heated overnight, the phases are separated, and the aqueous phase is washed three times with 150 ml each of diethyl ether. Then, the aqueous phase is acidified with 40 ml of concentrated H₂SO₄ in 200 ml of ice and extracted three times with 300 ml each of CH₂Cl₂. The collected organic phases are dried on MgSO₄ and the solvent is drawn off. A thin, highly liquid, light yellow oil is obtained.

### 4-Benzyloxybutanoic acid-t-butyl Ester

35 g (0.180 mol) of 4-benzyloxybutanoic acid and 20 ml of CH₂Cl₂ are filled in an autoclave and cooled to −70° C. (dry ice/acetone). Then, 90 ml of liquid isobutene and 2 ml of concentrated H₂SO₄ are added as catalyst. The autoclave is sealed and stirred for 72 hours at room temperature. After the pressure decreases, the brownish solution is diluted with 50 ml of CH₂Cl₂. This solution is first neutralized with KHCO₃ solution, then washed with citric acid solution and water. The milky, cloudy solution is dried on MgSO₄. After the solvent is drawn off, a light yellow oil is obtained.

### Di-(4-tert-butoxy-4-oxobutyl)malonate

This reaction is anhydrous and is performed in a nitrogen atmosphere. 12.30 g (76.80 mmol) of 4-hydroxybutyric acid-tert-butyl ester and 6.08 g (76.80 mmol) of dry pyridine are dissolved in 250 ml of absolute methylene chloride and cooled to 0° C. while being stirred. Then, 5.40 g (38.40 mmol), dissolved in 10 ml of absolute methylene chloride) of malonyl dichloride is slowly added. It is allowed to stir for two hours at 0° C. and for 12 hours at room temperature. Then, the reaction mixture is filtered on silica gel, washed with water, and the organic phase is dried on MgSO₄. The solvent is drawn off, and the product is purified by FC (cyclohexane:ethyl acetate 5:1). 9.70 g of a yellow, oily liquid is obtained.

### Di-(4-hydroxy-4-oxobutyl)malonate

15 ml of 98% formic acid is added to 1.57 g of di-(4-tert-butoxy-4-oxobutyl)malonate (4.04 mmol). The ester is dissolved and is completely deprived of protection after two days of stirring at room temperature. After the formic acid is removed in an oil pump vacuum, the dioic acid is obtained in a quantitative yield.

### 4-Nitro-4-[2-t-(butoxycarbonyl)ethyl]-heptanedioic Acid Diester

A solution of 16.2 ml of nitromethane (18.3 g, 0.3 mol) and three ml of triton B (40% in methanol) in 60 ml of dimethoxyethane is heated to 70° C. while being stirred. Then, 135 ml (119.2 g, 0.93 mol) of acrylic acid-t-butyl ester is added in drops, whereby the temperature is kept constant. After cooling is done subsequently, a total of six ml of triton B is added again within five minutes. Then, the reaction mixture is stirred for one hour at 70° C., then concentrated by evaporation in a vacuum and taken up in 600 ml of CH₂Cl₂. The solution is first washed with 10% HCl, then three times with saturated NaCl solution, and the organic phase is dried on MgSO₄. After the solvent is drawn off, the yellowish residue is recrystallized from ethanol. A colorless crystalline solid is obtained.

### 4-Amino-4-[2-t-(butoxycarbonyl)ethyl]-heptanedioic acid diester

25 g (60 mmol) of the first-generation nitro compound is dissolved while being heated gently in 600 ml of ethanol, and it is hydrogenated with 25 g of Raney nickel at room temperature and normal pressure. The reaction is monitored by means of TLC (SiO₂:hexane/EtOAc 1:2). After 48 hours, the catalyst is filtered off on Celite, and the solvent is removed in a vacuum. The yellow, oily residue is purified by column chromatography on silica gel (SiO₂:hexane/EtOAc 2:1). A white powder is obtained.

### 4-Nitro-[2-(carboxyethyl)]heptanedioic Acid

15 g (45 mmol) of NO₂[G-1] is dissolved in 100 ml of formic acid and stirred for 24 hours at room temperature. After about 30 minutes, a white precipitate begins to be deposited. The formic acid is drawn off in a vacuum. The white solid is then mixed three times with toluene, which in each case is distilled off again. After drying, a white powder is obtained.

### 9-Cascade:Nitromethane[3]:(2-Aza-3-oxypentylidyne):Propionic Acid-tert-butyl Ester

4.5 g (16 mmol) of 4-nitro-[2-(carboxyethyl)]heptanedioic acid, 22.3 g (53.5 mmol) of 4-amino-4-[2-t-(butoxycarbonyl)ethyl]-heptanedioic acid diester and 6.5 g (48 mmol) of 1-hydroxy-benzotriazole are dissolved in 350 ml of absolute DMF, and this solution is mixed with 9.92 g (48 mmol) of dicyclohexylcarbodiimide (DCC), dissolved in 50 ml of absolute DMF. After about one hour, a white precipitate begins to form. The reaction is monitored by means of TLC (SiO₂:hexane/EtOAc: 2:1). After 60 hours, precipitated DCU is filtered out, and the solvent is distilled off. The yellowish residue is taken up in 500 ml of ethyl acetate and washed in succession with 10% HCl, water, 10% NaHCO₃ solution and saturated NaCl solution. The organic phase is dried on MgSO₄, and the solvent is removed in a vacuum. After column-chromatographic purification on silica gel (SiO₂:hexane/EtOAc 2:1), a white powder is obtained.

### 9-Cascade:Aminomethane[3]:(2-Aza-3-oxypentylidyne):Propionic Acid-tert-butyl Ester

9.0 g (6 mmol) of the 2^{nd} generation nitro compound is dissolved in 100 ml of ethanol and hydrogenated with 5 g of Raney nickel at room temperature and normal pressure. After 48 hours, the catalyst is filtered off on Celite, and the solvent is drawn off. The yellowish residue is purified by (SiO₂:hexane/EtOAc 1:2), and the product is finally eluted with MeOH from the column. The solvent is removed in a vacuum. The residue is dissolved in CH₂Cl₂, and insoluble silica gel is filtered out. After the solvent is distilled off in a vacuum, a white solid that foams while drying is obtained.

### 18-Cascade: Dihydromethane[2]:(2-Aza-7-oxa-3,8-dioxooctylidyne):(2-Aza-3-oxopentylidyne):Propanoic Acid-tert-butyl Ester

A solution of 436 mg (1.580 mmol) of di-(4-hydroxy-4-oxobutyl)malonate, 716 mg (3.470 mmol) of DCC, 550 mg (3.470 mmol) of 1-HOBT and 5.00 g (3.47 mmol) of 2^{nd}-generation amine in DMF is stirred for 48 hours at room temperature in a moisture-free environment. After the precipitated DCU is filtered off and after the DMF is removed in a vacuum, the residue is dissolved in ethyl acetate and washed with 10% citric acid, water, 8% NaHCO₃ solution and saturated NaCl solution. Then, the organic phase is dried with MgSO₄, and the solvent is spun off. After FC (cyclohexane:ethyl acetate 1:1), a white solid is obtained.

### 18-Cascade: 1,2-Methano-1,2-dihydro[60]fullerene[2]:(2-Aza-7-oxa-3,8-dioxooctylidyne):(2-Aza-3-oxopentylidyne):Propanoic Acid-tert-butyl Ester

A solution of 439 mg (0.610 mmol) of N@C₆₀, 102 mg (0.670 mmol) of DBU, 202 mg (0.610 mmol) of CBr₄ and 1.90 g (0.61 mmol) of 18-cascade:dihydromethane[2]:(2-aza-7-oxa-3,8-dioxooctylidyne):(2-aza-3-oxopentylidyne):propanoic acid-tert-butyl ester in 300 ml of toluene (saturated with N₂) is allowed to stir for one day at room temperature. The reaction mixture is filtered on a silica gel column. First, unreacted N@C₆₀ is eluted with toluene, then the mixture that consists of mono- and bis-adducts is eluted with toluene:ethyl acetate 1:1. This mixture is separated with FC (ether:hexane 6:1), and a brown solid is obtained.

### 18-Cascade: 1,2-Methano-1,2-dihydro[60]fullerene[2]:(2-Aza-7-oxa-3,8-dioxooctylidyne):(2-Aza-3-oxopentylidyne):Propanoic Acid 8

0.648 g (0.17 mmol) in 98% formic acid is dissolved and stirred for 24 hours at room temperature. After the formic acid is removed in a vacuum, a red-brown powder is obtained.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

In the foregoing and in the following examples, all temperatures are set forth uncorrected in degrees Celsius and, all parts and percentages are by weight, unless otherwise indicated.

The entire disclosures of all applications, patents and publications, cited herein and of corresponding German application No. 102005007223.2, filed Feb. 15, 2005 and U.S. Provisional Application Ser. No. 60/653,488, filed Feb. 17, 2005, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.
[ⁱ] [Golman, K.; Leunbach, I.; Ardenjaer-Larsen, J. H.; Ehnholm, G. J.; Wistrand, L. G.; Petersson, J. S.; Järvi, A., and Vahasalo, S., Overhauser-Enhanced MR Imaging (OMRI), Acta Radiologica, 39, pp. 10-17 (1998)]
[ⁱⁱ] [Ardenkjaer-Larsen, J. H.; Laursen, I.; Leunbach, I.; Ehnholm, G.; Wistrand, L.-G.; Petersson, J. S.; Golman, K., EPR and DNP Properties of Certain Novel Single Electron Contrast Agents Intended for Oximetric Imaging, JMR 133, 1-12 (1998)]
[ⁱⁱⁱ] [Golman, K.; Petersson, J. S.; Ardenkjaer-Larsen, J. H.; Leunbach, I.; Wistrand, L. G.; Ehnholm, G. and Liu Kecheng, Dynamic in Vivo Oxymetry Using Overhauser Enhanced MR Imaging, J. Magn. Reson. Imag., 12, pp. 929-938 (2000)]
[^{iv}] [U. Katscher and S. Petersson, Kemspintomographie unter Nutzung des Overhausereffekts [Nuclear Spin Tomography with Use of Overhauser Effects], Phys. Bl. 56, pp. 51-54 (2000)
[^{v}] [Krishna, M. C.; English, S.; Yamada, K.; Yoo, J.; Murugesan, R.; Devasahayam, N.; Cook, J. A.; Golman, K.; Ardenkjaer-Larsen, J. H.; Subramanian, S.; Mitchell, J. B., Overhauser-Enhanced Magnetic Resonance Imaging for Tumor Oximetry: Coregistration of Tumor Anatomy and Tissue Oxygen Concentration, PNAS 99, pp. 2216-22 (2002)]
[^{vi}] [Krishna, M. C.; Subramanian, S.; Kuppusamy, P. and Mittchell, J. B., Magnetic Resonance Imaging for In Vivo Assessment of Tissue Oxygen Concentration. Semin Radiat Oncol, 1, pp. 58-69 (2001)]
[^{vii}] X. Camps; A. Hirsch, J. Chem. Soc. Perkin Trans. 1997, 11, 1595-1596.
[^{viii}] I. Lamparth; H. Karfunkel; A. Hirsch, Angew. Chem. [Applied Chemistry] 1994, 106, 453-455.
[^{ix}] I. Lamparth; H. Karfunkel; A. Hirsch, Angew. Chem. Int. Ed. 1994, 33, 437-438.
[^{x}] I. Lamparth; A. Hirsch, J. Chem. Soc., Chem. Commun. 1994, 14, 1727-1728
[^{xi}] [C. N. Murthy and K. E. Geckeler, The Water-Soluble □-Cyclodextrin-[60]Fullerene Complex, in: Chemical Communications, Vol. 13 (2001), pp. 1194-1195]
[^{xii}] A. Weidinger; T. Almeida Murphy; B. Mertesacker; M. Hohne, T. Pawlik, J.-M. Spaeth and B. Pietzak, Verfahren und Vorrichtung zur Herstellung von stabilen endohedralen Fullerenen der Struktur (Z@Cₓ) mit x≧60 [Process and Device for the Production of Stable Endohedral Fullerenes of Structure (Z@Cₓ) with x≧60, WO 98/00363
[^{xiii}] Goedde, B.; Waiblinger, M.; Dinse, K.-P.; Weidinger, A. Preparation of Highly-Enriched N@C60/C60 and N@C70/C70. Proceedings—Electrochemical Society (2001), 2001-11 (Fullerenes—Volume 11: Fullerenes for the New Millenium), 304-312.
[^{xiv}] U. Reuther; T. Brandmuller, et al., Chem. Eur. J. 2002, 8(10), 2261-2273.

1. Contrast medium for nuclear spin tomography with use of the Overhauser effect (OMRI), characterized in that
the contrast medium consists of endohedral fullerenes (Z@Cₓ)—Rₙ,
whereby Z means nitrogen or phosphorus,
R means a hydrophilic group,
N stands for a number between 1-10, and
X is a number between 60 and 82, as well as their physiologically compatible salts.

2. Contrast medium according to claim 1, wherein
R means a C(COY)₂ group, which is connected to the fullerene via two adjacent C atoms, and thus forms a cyclopropane ring, and Y, independently of one another, means NR¹R² or OR¹,
whereby R¹ and R², independently of one another, mean H, or C₁-C₁₀-alkyl, which is substituted with up to 6 hydroxyl groups, and
n stands for a number 1-10,
as well as their physiologically compatible salts.

3. Contrast medium according to claim 1, wherein
R means a C(COY)₂ group, which is connected to the fullerene via two adjacent C atoms and thus forms a cyclopropane ring,
Y means OR³, and
n is equal to 1,
whereby
R³ is a dendrimeric branched alkyl radical that comprises up to 50 C atoms, which can be interrupted by up to 10 N and/or O atoms or —C(O)N(H) radicals, and which can be substituted with up to 10 hydroxy-, carboxylic acid-, or carboxylic acid amide groups,
as well as their physiologically compatible salts.

4. Process for the production of the compounds according to claim 1, wherein hydrophilic functional groups are coupled covalently to an endohedral fullerene (Z@Cₓ), whereby Z and x are defined as in claim 1.

5. Use of contrast media according to claims 1 to 3 for performing oximetry measurements, wherein first a contrast medium with atomic nitrogen is used as an inclusion element (N@Cₓ)—Rₙ , and a first nuclear spin tomography is performed and taken at different times, and then a contrast medium with atomic phosphorus is used as an inclusion element (P@Cₓ)—Rₙ , and a second nuclear spin tomography is performed.
